(19) United States
(12) Patent Application Publication
(10) Pub. No.: US 2024/0392373 A1
(43) Pub. Date: Nov. 28, 2024

(54) Genetic Variants Associated with Opioid Use Disorder

(71) Applicant: Office of the Ohio Attorney General, Columbus, OH (US)

(72) Inventor: Jon E. Sprague, Columbus, OH (US)

(73) Assignee: Office of the Ohio Attorney General, Columbus, OH (US)

(21) Appl. No.: 18/672,523

(22) Filed: May 23, 2024

Related U.S. Application Data
(60) Provisional application No. 63/469,092, filed on May 26, 2023.

(51) Int. Cl. C12Q 1/6883

(52) U.S. Cl. CPC C12Q 1/6883; C12Q 2600/118; C12Q 2600/156

(57) ABSTRACT

Methods and kits for diagnosing a likelihood of developing opioid use disorder (OUD) using single nucleotide polymorphisms (SNPs) are described. Also described is a method of predicting a likelihood of a subject experiencing an opioid overdose.

### RELATED APPLICATIONS

This application claims priority to U.S. Provisional Application No. 63/469,092 filed under 35 U.S.C. § 111 (b) on May 26, 2023, the disclosure of which is incorporated herein by reference in its entirety.

### STATEMENT REGARDING FEDERALLY SPONSORED RESEARCH

This invention was made with no government support. The government has no rights in this invention.

### BACKGROUND

The opioid epidemic is claiming lives at an alarming rate, with a mortality rate exceeding that of motor vehicle accidents, HIV, or gun-related deaths. Opioid use disorder (OUD) is a serious public health issue that affects millions of people worldwide.

OUD is caused by a complex interplay of genetic, environmental, and social factors, and can be difficult to diagnose and treat. Some relatively limited gains have been made in OUD treatment and overdose prevention for people already suffering from OUD. However, this will not stem the tide of new cases of OUD.

Research about the risk of developing OUD is shockingly limited. Behavioral and socioeconomic factors associated with OUD have been identified, but there are no clinical tools with sufficient validation and predictive power to guide clinical decision making. This is particularly so for key settings such as emergency departments, where clinicians are tasked with treating acute pain in patients without an established relationship and often with limited information about the patient. The interaction between behavior, environment, and genetic factors is even less understood. Up to 50% of addiction can be attributed to genetics, but the technology, methods, and procedures to translate this knowledge into clinical practice have not been developed.

There is an immediate and critical need to develop methods to estimate each person's own unique risk for developing OUD prior to deciding to initiate opioid therapy for the management of their pain. There is currently no reliable method for diagnosing the disorder based on the presence of biomarkers.

The Diagnostic and Statistical Manual of Mental Disorders (DSM-5) and the WHO ASSIST 3.1 criteria are commonly used to self-identify aberrant behaviors, misuse, abuse, or dependence. These diagnostic tools assist in identifying when a patient is currently abusing prescription medications. However, these tools can take too much time to apply for practical clinical use and have been shown to have low sensitivity. Thus, there is a need to develop a prognostic tool for risk stratification prior to prescribing opioids.

### SUMMARY

Provided is a method of diagnosing a likelihood of opioid use disorder (OUD) in a patient, the method comprising (a) obtaining a biological sample from the patient; (b) isolating DNA from the biological sample; (c) genotyping the DNA for one or more single nucleotide polymorphisms (SNPs), wherein the one or more SNPs include rs15524, rs776746, rs324029, rs2654754, rs2740574, or rs2069514; (d) determining the presence or absence of the one or more SNPs in the isolated DNA; and (e) diagnosing the patient's likelihood of developing OUD based on the presence or absence of the one or more SNPs, wherein: a presence of any of rs15524, rs776746, rs324029, or rs2654754 is indicative of an increased risk of developing OUD, and a presence of either of rs2740574 or rs2069514 is indicative of a decreased risk of developing OUD. In certain embodiments, the biological sample is a buccal swab or oral fluid. In certain embodiments, the one or more SNPs are a combination of at least two of rs15524, rs776746, rs324029, rs2654754, rs2740574, or rs2069514.

In certain embodiments, the method further comprises administering a treatment to the patient based on the patient's diagnosed likelihood of developing OUD.

In certain embodiments, the method further comprises (f) administering to the patient a non-opioid pain management treatment if the patient has any of rs15524, rs776746, rs324029, or rs2654754, or administering to the patient an opioid for pain management if the patient has either of rs2740574 or rs2069514. In particular embodiments, the non-opioid pain management treatment is a non-opioid analgesic. In particular embodiments, the non-opioid pain management treatment is physical therapy, cognitive behavioral therapy, or a combination thereof.

In certain embodiments, the method further comprises (f) administering to the patient an opioid agonist or opioid antagonist if the patient has any of rs15524, rs776746, rs324029, or rs2654754. In particular embodiments, the opioid agonist is methadone or buprenorphine. In particular embodiments, the opioid antagonist is naltrexone, nalmefene, or naloxone. In particular embodiments, the method further comprises administering an opioid to the patient.

In certain embodiments, the absence of each of rs15524, rs776746, rs324029, and rs2654754 is indicative of no elevated risk of developing OUD. In certain embodiments, the absence of both of rs2740574 and rs2069514 is indicative of no decreased risk of developing OUD.

Further provided is a diagnostic kit for diagnosing a likelihood of a subject developing OUD based on the presence or absence of one or more SNPs, the diagnostic kit comprising: (a) a set of primers or probes for genotyping two or more of rs15524, rs776746, rs324029, rs2654754, rs2740574, and rs2069514 in DNA extracted from a biological sample; and (b) instructions for using the primers or probes to diagnose the subject's likelihood of developing OUD based on the presence or absence of the SNPs.

In certain embodiments, the set of primers or probes is for genotyping three or more of rs15524, rs776746, rs324029, rs2654754, rs2740574, and rs2069514 in DNA extracted from the biological sample. In certain embodiments, the set of primers or probes is for genotyping four or more of rs15524, rs776746, rs324029, rs2654754, rs2740574, and rs2069514 in DNA extracted from the biological sample. In certain embodiments, the set of primers or probes is for genotyping five or more of rs15524, rs776746, rs324029, rs2654754, rs2740574, and rs2069514 in DNA extracted from the biological sample. In certain embodiments, the set of primers or probes is for genotyping each of rs15524, rs776746, rs324029, rs2654754, rs2740574, and rs2069514 in DNA extracted from the biological sample.

Further provided is a computer-implemented method for diagnosing a likelihood of OUD in a patient, the method comprising: (a) receiving genotyping data for SNPs comprising rs15524, rs776746, rs324029, rs2654754, rs2740574, or rs2069514 in DNA extracted from a biological sample obtained from the patient; (b) processing the genotyping data to determine the presence or absence of the SNPs; and (c) using a computer algorithm to diagnose the patient's likelihood of developing OUD based on the presence or absence of the SNPs.

In certain embodiments, the computer algorithm comprises a machine learning algorithm trained on a dataset of genotyping data and clinical outcomes for OUD. In certain embodiments, the method comprises receiving genotyping data for SNPs comprising two or more of rs15524, rs776746, rs324029, rs2654754, rs2740574, and rs2069514. In certain embodiments, the method comprises receiving genotyping data for SNPs comprising three or more of rs15524, rs776746, rs324029, rs2654754, rs2740574, and rs2069514. In certain embodiments, the method comprises receiving genotyping data for SNPs comprising four or more of rs15524, rs776746, rs324029, rs2654754, rs2740574, and rs2069514. In certain embodiments, the method comprises receiving genotyping data for SNPs comprising four or more of rs15524, rs776746, rs324029, rs2654754, rs2740574, and rs2069514. In certain embodiments, the method comprises receiving genotyping data for SNPs comprising five or more of rs15524, rs776746, rs324029, rs2654754, rs2740574, and rs2069514. In certain embodiments, the method comprises receiving genotyping data for SNPs comprising each of rs15524, rs776746, rs324029, rs2654754, rs2740574, and rs2069514.

Further provided is a method of screening a population for a likelihood of OUD, the method comprising (a) obtaining a biological sample from each individual in the population; (b) isolating DNA from each biological sample; (c) genotyping the DNA for SNPs comprising rs15524, rs776746, rs324029, rs2654754, rs2740574, or rs2069514; (d) determining the presence or absence of the SNPs for each individual; and (e) identifying individuals in the population who are at increased or decreased likelihood of developing OUD based on the presence or absence of the SNPs, wherein a presence of any of rs15524, rs776746, rs324029, or rs2654754 is indicative of an increased risk of developing OUD, and a presence of either of rs2740574 or rs2069514 is indicative of a decreased risk of developing OUD. In certain embodiments, the method comprises genotyping the DNA for SNPs comprising two or more of rs15524, rs776746, rs324029, rs2654754, rs2740574, and rs2069514. In certain embodiments, the method comprises genotyping the DNA for SNPs comprising three or more of rs15524, rs776746, rs324029, rs2654754, rs2740574, and rs2069514. In certain embodiments, the method comprises genotyping the DNA for SNPs comprising four or more of rs15524, rs776746, rs324029, rs2654754, rs2740574, and rs2069514. In certain embodiments, the method comprises genotyping the DNA for SNPs comprising five or more of rs15524, rs776746, rs324029, rs2654754, rs2740574, and rs2069514. In certain embodiments, the method comprises receiving genotyping data for SNPs comprising each of rs15524, rs776746, rs324029, rs2654754, rs2740574, and rs2069514.

Further provided is a method of treating a patient diagnosed with a likelihood of OUD based on the presence of one or more particular SNPs, the method comprising (a) administering to the patient a therapeutically effective amount of an opioid agonist or opioid antagonist; and (b) adjusting a dosage of the opioid agonist based on the patient's genotyping results and odds ratio for OUD for rs15524, rs776746, rs324029, rs2654754, rs2740574, and rs2069514, wherein the dosage is decreased when any of rs15524, rs776746, rs324029, or rs2654754 is present, and the dosage is increased when either of rs2740574 or rs2069514 is present. In certain embodiments, the opioid agonist or antagonist is naloxone, naltrexone, nalmefene, methadone, buprenorphine, or a combination thereof.

Further provided is a method of preventing OUD in a patient diagnosed with a likelihood of OUD based on the presence of one or more particular SNPs, the method comprising (a) providing the patient with a personalized risk assessment based on the patient's genotyping results for rs15524, rs776746, rs324029, rs2654754, rs2740574, and rs2069514; and (b) prescribing a non-opioid alternative drug or an alternative non-opioid pain management treatment to the patient if the patient has any of rs15524, rs776746, rs324029, or rs2654754; or (c) prescribing an opioid to the patient if the patient has either of rs2740574 or rs2069514.

In certain embodiments, the alternative non-opioid pain management treatment is selected from the group consisting of physical therapy, cognitive behavioral therapy, and a combination thereof. In certain embodiments, the non-opioid alternative drug is a non-opioid analgesic.

Further provided is a method of diagnosing a likelihood of OUD in a subject, the method comprising asking the subject diagnostic questions; obtaining a biological sample from the subject; isolating DNA from the biological sample; genotyping the DNA for one or more SNPs comprising rs15524, rs776746, rs324029, rs2654754, rs2740574, or rs2069514; determining the presence or absence of the one or more SNPs in the DNA; and diagnosing a likelihood of OUD based on the determined presence or absence of the one or more SNPs combined with answers to the diagnostic questions. In certain embodiments, the one or more SNPs comprises rs324029.

Further provided is a method of determining a subject's likelihood of developing OUD, the method comprising obtaining a biological sample from the subject; isolating DNA from the biological sample; genotyping the DNA for the presence of a homozygous SNP in the CYP3A5 gene or the DRD3 gene; determining whether the homozygous SNP in the CYP3A5 gene or the DRD3 gene is present in the DNA; and determining the subject is likely to develop OUD if the homozygous SNP in the CYP3A5 gene or the DRD3 gene is present in the DNA. In certain embodiments, the homozygous SNP in the CYP3A5 gene is either rs15524 or rs776746. In certain embodiments, the homozygous SNP in the DRD3 gene is either rs324029 or rs2654754. In certain embodiments, the method further comprises administering to the subject a non-opioid pain management treatment.

Further provided is a method of predicting a likelihood of a subject experiencing an opioid overdose, the method comprising obtaining a biological sample from the subject; isolating DNA from the biological sample; genotyping the DNA for single nucleotide polymorphisms (SNPs) comprising rs776746, rs4436578, and rs6715729; determining the presence or absence of the SNPs in the DNA; and diagnosing an increased risk of the subject experiencing an opioid overdose if rs776746 or rs4436578 is present in the DNA, and diagnosing a decreased risk of the subject experiencing an opioid overdose if rs6715729 is present in the DNA. In certain embodiments, the method further comprises treating the subject with a non-opioid treatment. In certain embodiments, the method further comprises treating the subject with a reduced dose of an opioid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Flow diagram detailing the screening enrollment for the patient study described in the examples herein.

FIG. 2: Table 3, showing demographic data of patients with and without a diagnosis of OUD.

FIG. 3: Table 4, showing SNPs associated with OUD.

FIG. 4: Forest plot of individual odds ratios for significant rsIDs from Table 2.

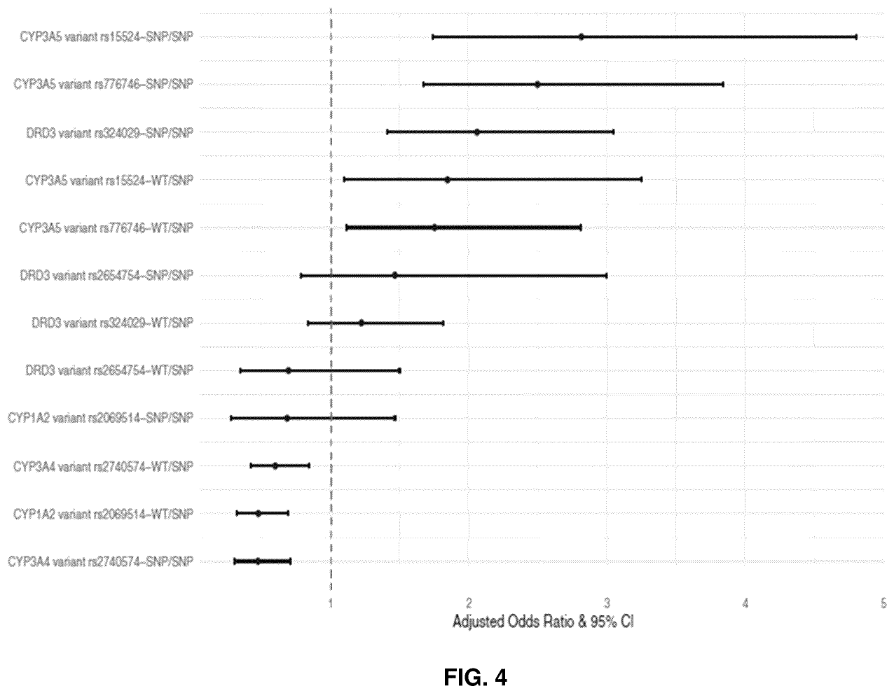

FIG. 5: P-value histogram for analyzing the results of the study described in the examples herein. The red line indicates the threshold of higher criticism.

FIG. 6: Table 5, showing SNP population frequencies of certain SNPs within the European and African American/Afro-Caribbean genetic ancestry groups.

FIG. 7: Learning curve procedure for analysis. Using DSM-5 criteria, 183 participants met the criteria for current OUD. A balanced training set of 122 current OUD (top boxes) and 122 non-OUD (bottom boxes) was utilized.

FIG. 8: Forest plot of individual odds ratios (reference: WT/WT) for significant rsIDs (Table 8). An rsID being significant means that at least one pairwise comparison between the three groups (SNP/SNP. WT/SNP, WT/WT) was statistically significant.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Without wishing to be bound by theory, it is believed that genetics contribute 30-40% to OUD, which allows for the possibility that genetic markers can be used to identify a personal risk for developing OUD. In accordance with the present disclosure, certain genetic markers in the form of single nucleotide polymorphisms (SNPs), or a combination of SNPs, can be used to assess an individual's risk for developing OUD, and the individual can be treated for pain management (or not treated at all, if appropriate) according to their determined OUD risk. This amounts to a paradigm shift whereby providers and patients can know the contribution, relative to other actors, of each individual's genetic risk for OUD prior to initiating opioid therapy. This can reduce human suffering and societal costs by improving pain management for some while reducing the risk of downstream opioid-related morbidity and mortality for others.

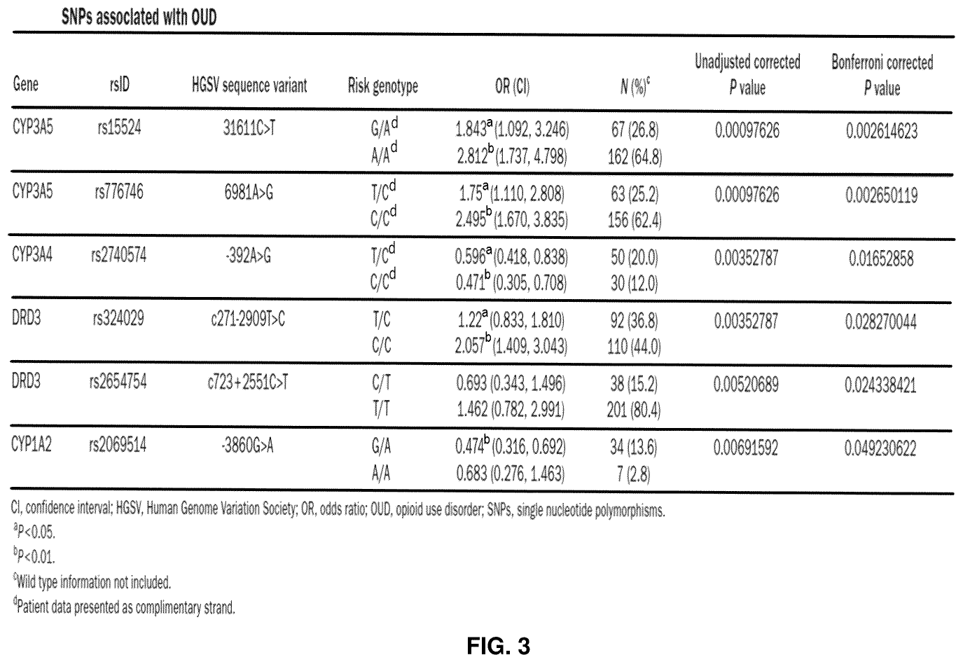

As described in the examples herein, the association between 180 candidate SNPs and OUD was assessed through a cross-sectional genetic association study where more than a thousand participants self-reported health history that included DSM-5 OUD criteria and contributed buccal swabs for testing of markers. The results validated a profile of genetic mutations associated with opioid addiction in a clinical setting by studying consenting emergency department patients in key centers throughout Ohio. Ohio is a state that has been hit especially hard by the opioid epidemic, ranking in the top five states for opioid overdose deaths, at a rate two- to three-times higher than the national average. Biological samples and self-reported factors potentially associated with OUD were collected. These included details of past opioid exposure, current indications of OUD, and other health history. The biological samples were used for testing an array of SNPs possibly associated with OUD risk. An identifiable genetic mutation profile independently associated with OUD risk was consequently uncovered by determining which gene markers are more frequently present in patients with moderate to severe OUD than in patients who do not have OUD, and by determining the extent to which these genetic associations are independent of other patient characteristics through an interview with the patient and a review of medical records. The results of the study identified six SNPs that are associated with OUD. These SNPs and associated genes are: CYP3A5 (rs15524 and rs776746), DRD3 (rs324029 and rs2654754), CYP3A4 (rs2740574), and CYP1A2 (rs2069514). (FIG. 3.) In particular, each of the SNPs rs15524, rs776746, rs324029, and rs2654754, when present, indicate an increased likelihood of the subject developing OUD, and each of the SNPs rs2740574 and rs2069514, when present, indicate a decreased likelihood of the subject developing OUD. In addition, the presence of a homozygous SNP in the CY3A5 gene (namely, rs15524 or rs776746) or DRD3 gene (rs324029 or rs2654754) indicates an increased likelihood of the subject developing OUD as compared to the heterozygotic SNP in these genes. (FIG. 3.) Accordingly, these SNPs may serve as biomarkers for assessing the risk of developing OUD in patients. Furthermore, it has been discovered that the presence of the rs776746 or rs4436578 SNP is indicative of a subject having an increased risk of experiencing an opioid overdose, and the presence of the rs6715729 SNP is indicative of a subject having a decreased risk of experiencing an opioid overdose. The risk profile may be assessed and viewed in combination with other behavioral and environmental risk factors by a clinician in determining an appropriate pain management strategy so as to maximize clinical pain management while minimizing the likelihood of OUD.

One method described herein is a diagnostic tool for assessing the likelihood of developing OUD based on screening tools to identify which patients are at risk for development of OUD when initiating opioid therapy. The method can be used in a variety of settings, including clinical settings, research settings, and forensic settings. The method can also be used in conjunction with other diagnostic tools and risk assessment methods to improve the accuracy of diagnosing and predicting OUD. The method may involve analyzing a sample from the patient for one or more SNPs, such as the SNPs rs15524, rs776746, rs324029, rs2654754, rs2740574, and rs2069514.

To perform the method, a biological sample is obtained from a patient. The sample can be any suitable biological material, such as blood, saliva, or tissue. The sample can be, for example, a buccal swab or oral fluid. The DNA is then extracted from the sample using standard techniques, such as phenol-chloroform extraction or commercial DNA extraction kits. The DNA is then genotyped for rs15524, rs776746, rs324029, rs2654754, rs2740574, and/or rs2069514 using any suitable genotyping technique, such as PCR-based methods, microarray-based methods, or sequencing-based methods. The genotyping results are then used to determine the patient's likelihood of developing OUD.

The method may involve making an assessment or diagnosis of a likelihood of the patient developing OUD based on the presence or absence of the one or more SNPs, where the presence of any of rs15524, rs776746, rs324029, or rs2654754 indicates an increased likelihood of developing OUD, the presence of either rs2740574 or rs2069514 indicates a decreased likelihood of developing OUD, the absence of each of rs15524, rs776746, rs324029, and rs2654754 indicates a non-increased risk of developing OUD, and the absence of both of rs2740574 and rs2069514 indicates a non-decreased risk of developing OUD.

The method may further involve collecting and analyzing the patient's answers to one or more questions about certain variables which may be associated with OUD. Such questions may include, but are not limited to, questions about past opioid exposure, substance use, mental health disorders, prior trauma, pain, and other health history. Questions derived from the Diagnostic and Statistical Manual of Mental Health Disorders (DSM-5) may be asked. For example, patients who report having used opioids within the past 12 months may be asked questions derived from the DSM-5, while patients with reported opioid use longer than one year prior may be asked modified DSM-5 questions. A score of a certain threshold (such as 2 or higher) from the DSM-5 questions may result in categorizing the patient as meeting the criteria for OUD.

Once the patient's risk of developing OUD is determined, the patient may be treated in a manner that takes into account the determined risk of developing OUD. For example, a patient who is determined to have an increased risk of developing OUD may be treated for pain management with a non-opioid drug such as a non-opioid analgesic, or prescribed a non-opioid therapy such as physical therapy or cognitive behavioral therapy, or a combination thereof. Non-limiting examples of non-opioid analgesics include acetaminophen, ibuprofen, or a combination thereof. Alternatively, a patient who is determined to have an increased risk of developing OUD may be treated with an opioid in combination with a drug capable of reducing the risk of opioid addiction or dependence, such as an opioid agonist or antagonist. Non-limiting examples of such drugs include methadone, buprenorphine, and naltrexone. Alternatively, a patient who is determined to have an increased risk of developing OUD may be tapered off of opioids. Alternatively, a patient who is determined to have an increased risk of developing OUD may be treated for pain management by increasing a dose of an opioid agonist or antagonist, decreasing a dose of an opioid, increasing a dose of a non-opioid drug, or any combination thereof. A patient who is determined to have a decreased risk of developing OUD may be treated for pain management with an opioid drug, or by increasing the dose of an opioid drug, or by decreasing the dose of an opioid agonist or antagonist, or a combination thereof. However, a decreased risk of developing OUD does not mean no risk of developing OUD. It is known that prescribing opioids can lead to increased tolerance, dependence, and addiction. Therefore, the use of non-opioid therapies may still be considered.

The present disclosure provides a reliable and accurate method of diagnosing a likelihood of a subject developing OUD based on the presence or absence of one or more particular SNPs in the subject's DNA. This has a wide range of applications and can be used to improve the prevention, diagnosis, and treatment of OUD.

The present disclosure may also be made available via a kit containing one or more key components to practice the methods described herein. A non-limiting example of such a diagnostic kit includes a set of primers or probes for genotyping two or more SNPs selected from rs15524, rs776746, rs324029, rs2654754, rs2740574, and rs2069514 in DNA extracted from a biological sample, and instructions for using the primers or probes to diagnose a subject's likelihood of developing OUD based on the presence or absence of the SNPs in the extracted DNA. The primers or probes and instructions may be housed in separate containers, where the containers may or may not be present in a combined configuration. The instructions may be recorded on a suitable recording medium. For example, the instructions may be present in the kit as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example I—Patient Study

The influence of genetic variants related to opioid overdose was evaluated using multiple logistic regression analysis in self-reported, nonfatal opioid overdoses. The genetic association of 180 candidate SNPs in patients with the occurrence of OUD was evaluated to find associations between the candidate SNPs and OUD. Out of the 180 candidate SNPs, 120 related to the dopamine reward pathway and 60 related to pharmacokinetics, specifically drug metabolism or transport.

Participants were randomly recruited in a cross-sectional genetic association study. Self-reported health history including DSM-5 OUD criteria and buccal swabs were collected. A total of 1,301 adult participants (≥18 years of age) seen in emergency departments of three medical centers in Ohio were included in this study. Of the 821 patients who reported having been exposed to opioids in their lifetime, 95 (11.6%) also reported having experienced an opioid-related overdose. Of the included participants, 250 met the DSM-5 criteria for ever having OUD. Logistic regression, adjusting for age and biologic sex, was used to characterize the association between each SNP and DSM-5 criteria consistent with OUD. Six SNPs found in 4 genes were each associated with OUD: increased odds with CYP3A5 (rs15524 and rs776746) and DRD3 (rs324029 and rs2654754), and decreased odds with CYP3A4 (rs2740574) and CYP1A2 (rs2069514). Homozygous SNPs in CYP3A5, rs15524 (A/A) and rs776746 (C/C) had the highest adjusted odds ratio of 2.812 (95% confidence interval (CI) 1.737, 4.798) and 2.495 (95% CI 1.670, 3.835), respectively. Additionally, two homozygous SNPs in DRD3, rs324029 (C/C) and rs2654754 (T/T), with odds ratios of 2.057 and 1.462, respectively, are also related to increased risk of OUD. Variants within the dopamine reward and opioid metabolism pathways have significant positive (DRD3 and CYP3A5) and negative (CYP3A4 and CYP1A2) associations with OUD. Identification of these variants allows for genetic prognostication, risk assessment, and customization of therapeutic strategies.

### Study Design

This cross-sectional genetic association study utilized a random selection model to guide screening of consecutive patients within three large urban emergency departments (EDs) in Ohio, a state which has ranked in the top 5 for opioid overdose deaths since 2014. All English-speaking patients aged 18 years or older with the capacity to consent were eligible for enrollment, which took place over a 13-month period. The study involved cross-sectional data collection of randomly approached patients during an energy department encounter. Enrollment took place at two academic health systems encompassing three urban EDs. This study was approved by the institutional review boards of the participating institutions. After informed consent was obtained, participants completed an in-depth survey assessment to collect self-reported variables potentially associated with OUD, including details of past opioid exposure, substance use, mental health disorders, prior trauma, pain, and other health history. Any participant who reported use of opioids within the past 12 months was then asked questions derived from the DSM-5 criteria for OUD to determine current OUD. The questions were administered by research staff who were trained by a licensed psychologist to minimize the potential for misclassification bias. To determine whether participants may have met criteria for ever having OUD in their lifetime (i.e., at any point in their lives), those that reported opioid use, but longer than 1 year ago, were asked modified DSM-5 questions, with coaching to think back to whether the answer would be true within a 12-month time-span during which they were using opioids. Scores from the DSM-5 questions were tallied and anyone with a score of 2 or higher was categorized as meeting criteria for opioid use disorder, consistent with the use of DSM-5 scoring in clinical practice. Additionally, detailed self-reported race and ethnicity characteristics were then related to the biogeographical genetic ancestry group commonly reported in pharmacogenomics (PGx). Cheek cells were collected via buccal swab by the enrolling research team.

### Genotyping

Cheek cell samples were shipped to a Clinical Laboratory Improvement Amendments (CLIA) certified laboratory (Genemarkers LLC. Kalamazoo, MI) for genotyping of the 180 candidate SNPs (Table 1). The 180 candidate SNPs were analyzed using the laboratory's commercially available PGx panel containing 60 SNPs related to drug metabolism or transport (pharmacokinetics) and 120 candidate SNPs related to pharmacodynamics and the dopamine reward pathway including opioid receptors. These candidate SNPs were identified through a query of the PubMed database. DNA was isolated and processed as previously described. Strengthening the Reporting of Genetic Association (STREGA) guidelines were used in reporting the genetic association findings.

TABLE 1
Candidate SNP variant levels of evidence

| | | PharmGKB | CPIC | | |
|---|---|---|---|---|---|
| Gene.Symbol | NCBI.SNP.Reference | LOE^{a} | LOE^{a} | PMID^{c} | Note |
| ABCB1/MDR1 | rs1045642 | 3 | C | | |
| ANKK1 | rs2734849 | x | x | 25521358 | |
| ANKK1 | rs1800497 | 3 | x | | |
| ARHGAP28 | rs2567261 | x | x | 4233207 | |
| AUTS2 | rs6943555 | x | x | 25398668 | |
| BDNF | rs6265 | 4 | x | | |
| BDNF | rs11030104 | x | x | 27647760 | |
| BDNF | rs10767664 | x | x | 27647760 | |
| BDNF | rs16917234 | 3 | x | | |
| BDNFOS/antiBDNF | rs988712 | x | x | 27647760 | |
| BDNFOS/antiBDNF | rs7481311 | x | x | 27647760 | |
| BDNFOS/antiBDNF | rs11030096 | 3 | x | | |
| CACNA2D2 | rs5030977 | x | x | 25247900 | |
| CNIH3 | rs1369846 | x | x | 26239289 | |
| CNIH3 | rs1436171 | x | x | 26239289 | |
| CNIH3 | rs1436175 | x | x | 26239289 | |
| CNIH3 | rs10799590 | 3 | x | | |
| CNIH3 | rs12130499 | x | x | 26239289 | |
| CNIH3 | rs298733 | x | x | 26239289 | |
| CNR1 | rs2023239 | 3 | x | | |
| CNR1 | rs806379 | x | x | 16741937 | |
| CNR1 | rs6928499 | 3 | x | | |
| COMT | rs4680 | 3 | C | | |
| CREBBP | rs3025684 | x | x | 27699938 | |
| CSNK1E | rs1534891 | 3 | x | | |
| CSNK1E | rs6001093 | x | x | 25521358 | |
| CSNK1E | rs135745 | x | x | 25521358 | |
| CYP1A2 | rs2069514 | | | 21593735 | Related to OUD |
| | | | | | risk |
| CYP1A2 | rs35694136 | | | | Clinical panel^{d} |
| CYP1A2 | rs762551 | | | | Clinical panel |
| CYP1A2 | rs12720461 | | | | Clinical panel |
| CYP2B6 | rs3745274 | 2A | B | | |
| CYP2C19 | rs12248560 | | | | Clinical panel |
| CYP2C19 | rs4244285 | | | | Clinical panel |
| CYP2C19 | rs4986893 | 4 | x | | |
| CYP2C19 | rs28399504 | | | | Clinical panel |
| CYP2C19 | rs56337013 | | | | Clinical panel |
| CYP2C19 | rs72552267 | | | | Clinical panel |
| CYP2C19 | rs72558186 | | | | Clinical panel |
| CYP2C19 | rs41291556 | | | | Clinical panel |
| CYP2C19 | rs28399507 | | | | Clinical panel |
| CYP2C9 | rs28371685 | | | | Clinical panel |
| CYP2C9 | rs1799853 | 4 | x | | |
| CYP2C9 | rs1057910 | 4 | x | | |
| CYP2C9 | rs56165452 | | | | Clinical panel |
| CYP2C9 | rs28371686 | | | | Clinical panel |
| CYP2C9 | rs9332131 | | | | Clinical panel |
| CYP2D6 | rs5030862 | | | | Clinical panel |
| CYP2D6 | rs5030865 | | | | Clinical panel |
| CYP2D6 | rs28371706 | 1A | A | | |
| CYP2D6 | rs16947 | 1A | A | | |
| CYP2D6 | rs59421388 | 1A | A | | |
| CYP2D6 | rs1080985 | | | | Clinical panel |
| CYP2D6 | rs35742686 | 1A | A | | |
| CYP2D6 | rs769258 | | | | Clinical panel |
| CYP2D6 | rs3892097 | 1A | B | | |
| CYP2D6 | rs1065852 | 1A | A | | |
| CYP2D6 | rs28371725 | 1A | A | | |
| CYP2D6 | rs5030655 | 1A | A | | |
| CYP2D6 | rs5030867 | 2A | C | | |
| CYP2D6 | rs5030865 | | | | Clinical panel |
| CYP2D6 | hCV32407229 | | | | Clinical panel |
| CYP3A4 | rs12721629 | 3 | C | | |
| CYP3A4 | rs4987161 | 3 | C | | |
| CYP3A4 | rs2740574 | 3 | x | 29450233 | Related to OUD |
| | | | | | risk |
| CYP3A4 | rs55785340 | 3 | C | | |
| CYP3A4 | rs35599367 | 3 | C | | |
| CYP3A4 | rs4986910 | 3 | C | | |
| CYP3A5 | rs28365083 | | | | Clinical panel |
| CYP3A5 | rs15524 | x | x | 34523354 | Related to OUD |
| | | | | | risk |
| CYP3A5 | rs776746 | 3 | C | 22277678 | Related to OUD |
| | | | | | risk |
| CYP3A5 | rs28383468 | x | x | | Clinical panel |
| CYP3A5 | rs10264272 | 3 | x | | |
| CYP3A5 | rs41303343 | | | | Clinical panel |
| CYP3A5 | rs55817950 | | | | Clinical panel |
| CYP3A5 | rs28383479 | | | | Clinical panel |
| DBH | rs1611115 | 3 | x | | |
| DBI | rs12613135 | x | x | 26277529 | |
| DCC | rs2292043 | x | x | 27676367 | |
| DCC | rs12607853 | x | x | 27676367 | |
| DCC | rs16956878 | x | x | 27676367 | |
| DRD2 | rs2075652 | x | x | 26146874 | |
| DRD2 | rs2734833 | x | x | 26146874 | |
| DRD2 | rs4436578 | x | x | 26146874 | |
| DRD2 | rs1079596 | 3 | x | | |
| DRD2 | rs17115583 | x | x | 27819741 | |
| DRD2 | rs11214607 | x | x | 26146874 | |
| DRD2 | rs1125394 | 3 | x | | |
| DRD2 | rs1799978 | x | x | 26437921 | |
| DRD2 | rs2283265 | 3 | x | | |
| DRD2 | rs1076560 | 3 | x | | |
| DRD2 | rs6275 | 3 | x | | |
| DRD2 | rs6277 | 4 | x | | |
| DRD2/Tag1B | rs1079597 | x | x | 25521358 | |
| DRD3 | rs9825563 | x | x | 24398431 | |
| DRD3 | rs6280 | 3 | x | | |
| DRD3 | rs324029 | 3 | x | | Related to OUD |
| | | | | | risk |
| DRD3 | rs9288993 | 3 | x | | |
| DRD3 | rs2654754 | 3 | | | Related to OUD |
| | | | | | risk |
| DRD4 | rs1800955 | x | x | 23840506 | |
| EXOC4 | rs718656 | 3 | x | | |
| FAAH | rs2295633 | x | x | 25558980 | |
| FAAH | rs3766246 | 3 | x | | |
| FAAH | rs324420 | 3 | x | | |
| FSTL4 | rs31347 | x | x | 18195715 | |
| GABRB3 | rs4906902 | 3 | x | | |
| GABRB3 | rs7165224 | x | x | 26277529 | |
| GAD1 | rs2058725 | x | x | 26277529 | |
| GAD1 | rs1978340 | x | x | 26277529 | |
| GAL | rs948854 | 3 | x | | |
| GAL | rs694066 | 3 | x | | |
| GRIK1 | rs2832407 | x | x | 26441400 | |
| GRIN3A | rs17189632 | x | x | 27542340 | |
| GRIN3B | rs2240158 | x | x | 27542340 | |
| HRPT2/CDC73 | rs1408830 | x | x | 18195715 | |
| HTR2A | rs6311 | | | | Clinical panel |
| HTR2A | rs6313 | | | | Clinical panel |
| HTR2C | rs3813929 | | | | Clinical panel |
| HTR3B | rs1185027 | x | x | 27773795 | |
| HTR3B | rs11606194 | x | x | 25948405 | |
| HTR3B | rs3758987 | 3 | x | | |
| intergenic | rs1986513 | x | x | 18195715 | |
| intergenic | rs10494334 | x | x | 20520587 | |
| intergenic | rs966162 | x | x | 18195715 | |
| intergenic | rs965972 | x | x | 18195715 | |
| intergenic | rs952985 | x | x | 18195715 | |
| KCNC1 | rs60349741 | x | x | 24143882 | |
| LINC01456 | rs2213602 | x | x | 18195715 | |
| MPDZ | rs1389752 | | x | 26384852 | |
| MTHFR | rs1801131 | | | | Clinical panel |
| MTHFR | rs1801133 | | | | Clinical panel |
| NFKB1 | rs230530 | x | x | 26384852 | |
| NR4A2 | rs1405735 | x | x | 18195715 | |
| NRXN3 | rs11624704 | x | x | 25242908 | |
| NTSR1 | rs3915568 | x | x | 26384852 | |
| OPRD1 | rs4654327 | 3 | x | | |
| OPRD1 | rs6669447 | x | x | 28692418 | |
| OPRD1 | rs2236857 | 3 | x | | |
| OPRD1 | rs678849 | 3 | x | | |
| OPRD1 | rs2236861 | 3 | x | | |
| OPRD1 | rs10753331 | x | x | 22795689 | |
| OPRD1 | rs508448 | 3 | x | | |
| OPRD1 | rs1042114 | x | x | 22795689 | |
| OPRD1 | rs2234918 | 3 | x | | |
| OPRK1 | rs6473797 | 3 | x | | |
| OPRK1 | rs1051660 | 3 | x | | |
| OPRM1 | rs1799971 | 3 | C | | |
| OPRM1 | rs1074287 | x | C | | |
| OPRM1 | rs510769 | 3 | C | | |
| OPRM1 | rs1381376 | x | C | | |
| OPRM1 | rs563649 | x | C | | |
| OPRM1 | rs9479757 | 3 | C | | |
| OPRM1 | rs2075572 | 3 | C | | |
| OPRM1 | rs6848893 | 3 | C | | |
| PDYN | rs2235749 | x | x | 31510971 | |
| PDYN | rs910080 | 3 | x | | |
| PDYN | rs1997794 | 3 | x | | |
| PDYN | rs1022563 | 3 | x | | |
| PENK | rs2609997 | x | x | 26164485 | |
| RGS9-2 | rs1530351 | x | x | 25591550 | |
| SCN9A | rs6746030 | x | x | 27061230 | |
| SLC6A3 | rs2652511 | x | x | 20580759 | |
| SLCO1B1 | rs2306283 | x | x | 24086514 | |
| SLCO1B1 | rs4149056 | x | x | 23480028 | |
| SORCS3 | rs728453 | x | x | 18195715 | |
| TACR1 aka NK1R | rs6715729 | x | x | 19553914 | |
| TACR1 aka NK1R | rs735668 | 3 | x | | |
| TACR1 aka NK1R | rs6741029 | x | x | 19553914 | |
| TACR3 aka NK3R | rs1384401 | 3 | x | | |
| TACR3 aka NK3R | rs4530637 | 3 | x | | |
| TAOK3 | rs795484 | 3 | x | | |
| UGT2B7 | rs7668258 | 3 | x | | |
| UGT2B7 | rs7662029 | 3 | x | | |
| UGT2B7 | rs7438135 | 3 | x | | |
| VKORC1 | rs9923231 | | | | Clinical panel |
| WLS | rs1036066 | x | x | 23566366 | |
| WLS | rs983034 | x | x | 23566366 | |
| WLS | rs3748705 | x | x | 23566366 | |
| ZNF804A | rs7597593 | 3 | x | | |
| ZNF804A | rs1344706 | x | x | 25708696 | |

^{a}Pharmacogenomics Knowledgebase levels of evidence
^{b}Clinical Pharmacogenetic Implementation Consortium levels of evidence
^{c}PubMed reference number
^{d}Not necessarily related to opioid use

### Statistical Analysis

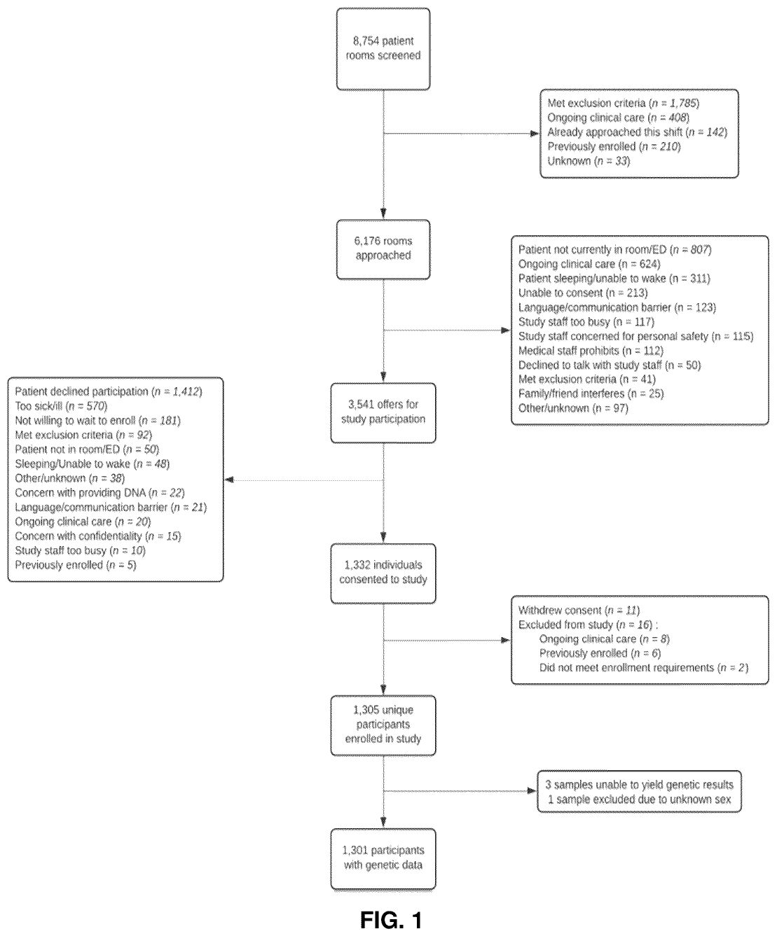

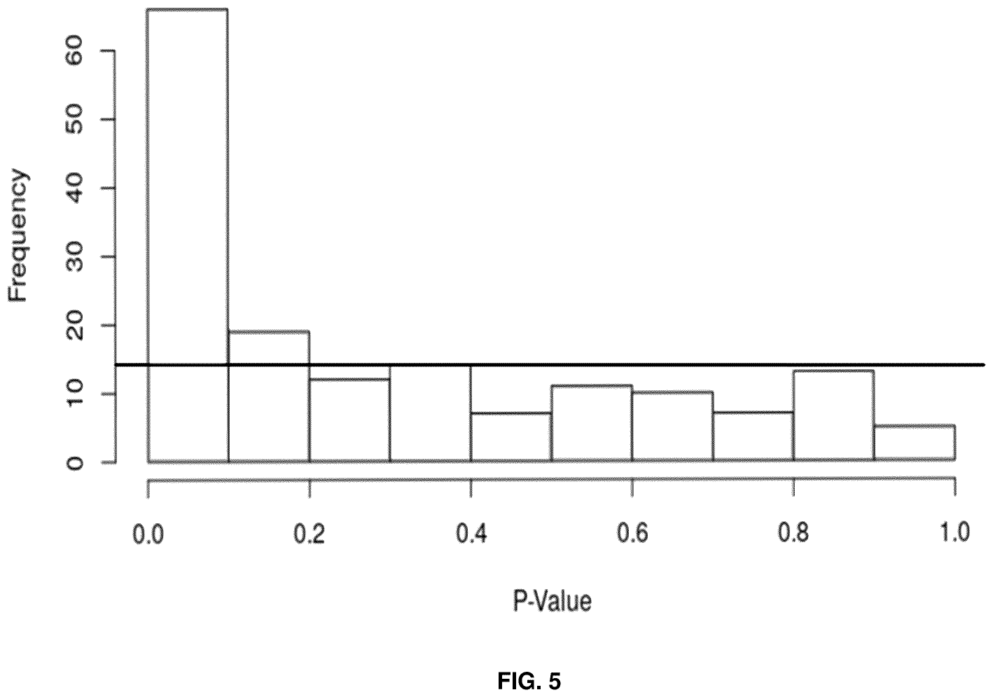

An a priori power analysis revealed that a sample size of 1,300 would have 80% power, at an alpha of 0.05, to detect an absolute difference of 1.4% in the prevalence of genetic risk factors between the general population and those with OUD (assuming a prevalence of genetic risk factors of 1% in controls and 2.4% in those with OUD). For each of the 180 candidate SNPs, a logistic regression was performed on the binary outcome of OUD, while controlling for the patient's reported age and biologic sex at birth. Race was not controlled for, recognizing that this variable is not an accurate representation of biologic variation in a heterogeneous society. A type-I (sequential) analysis of variance was completed to test whether the model with the candidate SNP, age, and sex is statistically superior compared with the model with just age and sex. This essentially tested the following hypothesis: while adjusting for age and sex, there is at least one difference among the three possible groups for the candidate SNP (SNP/SNP, wildtype (WT)/SNP, WT/WT). A P value histogram (from the P values from the type-I analysis of variance) was constructed to visualize the general distribution of the studied hypotheses. This histogram is shown in FIG. 5. For statistical inference, two strategies for adjusting for multiple comparisons were considered. To control the false discovery rate (FDR), the Benjamini and Hochberg FDR method was applied, whereas the Bonferroni correction method was used to control the family-wise error rate. The Hardy-Weinberg equilibrium was not evaluated because the study population does not meet the basic assumptions of Hardy-Weinberg equilibrium; namely, the SNPs investigated were preselected. These analyses were implemented in R version 4.0.4. Patients with missing genetic data were excluded from the analysis. Details of screening enrollment are shown in FIG. 1.

### Results

### Demographic Data

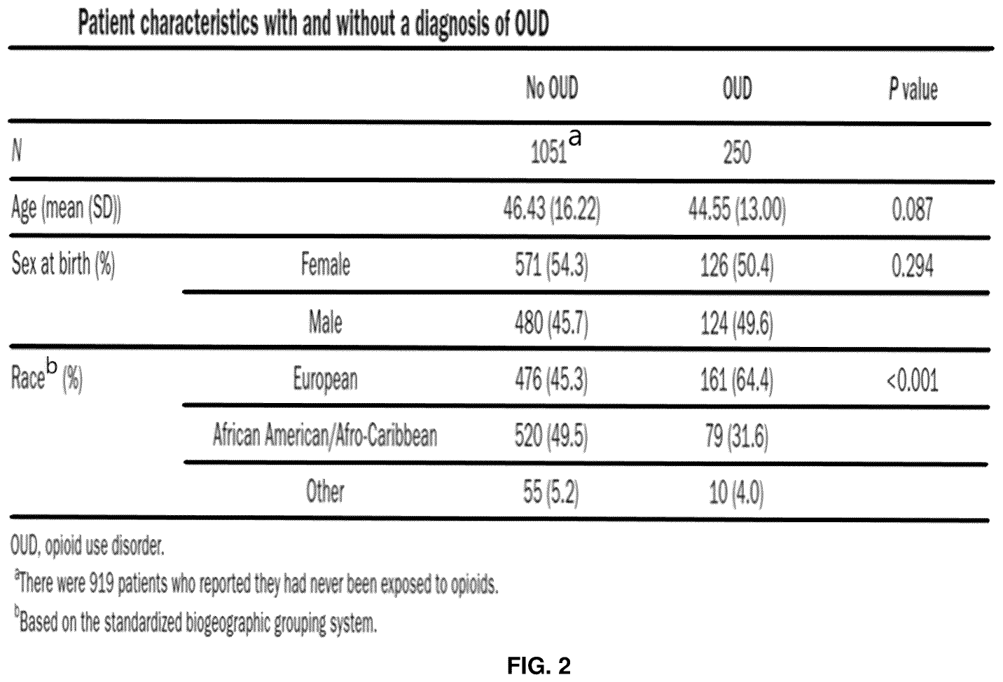

A total of 1,301 participants were included in the analyses for this study. Of included participants, 250 (126 women and 124 men) met the DSM-5 criteria for OUD, 64.4% of which were of the European genetic ancestry group and 31.6% were of the African American/Afro-Caribbean genetic ancestry group (Table 3, FIG. 2).

Genetic Variants Associated with Opioid Use Disorder

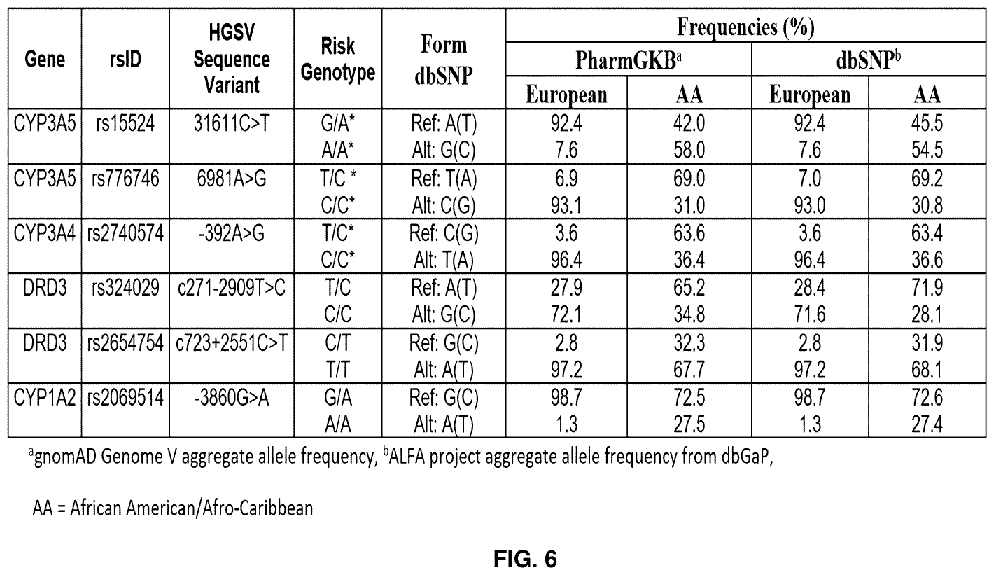

A total of 55 SNPs had uncorrected P values <0.05. A total of 28 SNPs had FDR corrected P values <0.05 (Table 2). A total of 6 SNPs had Bonferroni corrected P values <0.05. Here, the six SNPs found after applying the Bonferroni correction are discussed. The estimates and 95% confidence intervals (CIs) from the FDR can be found in Table 2. The estimates and 95% CIs following the Bonferroni correction are found in Table 4 (FIG. 3). The 6 SNPs identified as being associated with OUD came from a total of 4 genes: CYP3A5, DRD3, CYP3A4, and CYP1A2. Variants of CYP3A5 (rs15524 and rs776746) and DRD3 (rs324029 and rs2654754) were associated with increased odds (reference: WT/WT) of OUD, and variants of CYP3A4 (rs2740574) and CYP1A2 (rs2069514) were associated with decreased odds (reference: WT/WT; Table 4, FIG. 3). Homozygotic CYP3A5 (rs15524 and rs776746) had the highest odds ratios (ORs; CI) of 2.812 (95% CI 1.737, 4.798) and 2.495 (95% CI 1.670, 3.835), respectively (FIG. 2). The general population frequencies of these SNPs within the European and African American/Afro-Caribbean genetic ancestry groups can be found in Table 5 (FIG. 6).

TABLE 2
False discovery rate (FDR) for SNPs associated with OUD

| | | | | | | | | | Bonferroni |
|---|---|---|---|---|---|---|---|---|---|
| | | HGSV Sequence | Risk | Odds Ratio | | | | | Corrected |
| Gene | rsID | Variant | Geno-type | (Confidence Interval) | | N (%)^{c} | | FDR | p value |
| CYP3A5 | rs15524 | 31611C > T | G/A* | 1.843^{a} | (1.092, 3.246) | 67 | (26.8) | 0.00132506 | 0.002614623 |
| | | | A/A* | 2.812^{b} | (1.737, 4.798) | 162 | (64.8) | | |
| CYP3A5 | rs776746 | 6981A > G | T/C* | 1.75^{a} | (1.110, 2.808) | 63 | (25.2) | 0.00132506 | 0.002650119 |
| | | | C/C* | 2.495^{b} | (1.670, 3.835) | 156 | (62.4) | | |
| CYP3A4 | rs2740574 | −392A > G | T/C* | 0.596^{a} | (0.418, 0.838) | 50 | (20.0) | 0.005509527 | 0.01652858 |
| | | | C/C* | 0.471^{b} | (0.305, 0.708) | 30 | (12.0) | | |
| DRD3 | rs324029 | c271 − 2909T > C | T/C | 1.22^{a} | (0.833, 1.810) | 92 | (36.8) | 0.005654009 | 0.028270044 |
| | | | C/C | 2.057^{b} | (1.409, 3.043) | 110 | (44.0) | | |
| DRD3 | rs2654754 | c723 + 2551C > T | C/T | 0.693 | (0.343, 1.496) | 38 | (15.2) | 0.005654009 | 0.024338421 |
| | | | T/T | 1.462 | (0.782, 2.991) | 201 | (80.4) | | |
| CYP1A2 | rs2069514 | −3860G > A | G/A | 0.474^{b} | (0.316, 0.692) | 34 | (13.6) | 0.007647364 | 0.049230622 |
| | | | A/A | 0.683 | (0.276, 1.463) | 7 | (2.8) | | |
| CYP2D6 | rs16947 | *2*4k*8*11*14*17*29*41 + | G/A* | 0.719^{a} | (0.530, 0.972) | 98 | (39.2) | 0.007647364 | 0.053531545 |
| | | 2850C > T | AA* | 0.466^{b} | (0.289, 0.671) | 33 | (13.2) | | |
| BDNF | rs16917234 | c25 − 18242A > G | A/G | 0.459^{b} | (0.297, 0.685) | 29 | (11.6) | 0.007700938 | 0.069308446 |
| | | | G/G | 0.942 | (0.482, 1.721) | 13 | (5.2) | | |
| LINC01456 | rs2213602 | 18089955A > G | A/G | 0.646 | (0.396, 1.026) | 27 | (10.8) | 0.007700938 | 0.06876795 |
| | | | G/G | 0.542^{b} | (0.390, 0.745) | 61 | (24.4) | | |
| CYP3A5 | rs10264272 | *6 14690G > A | C/T* | 0.380^{b} | (0.190, 0.686) | 11 | (4.4) | 0.010394632 | 0.103946318 |
| | | | T/T* | 0.0 | | 0 | (0.0) | | |
| CYP2D6 | rs1065852 | *4*10*14A + | G/A* | 1.774^{b} | (1.304, 2.404) | 84 | (33.6) | 0.010577728 | 0.126932741 |
| | | 100C > T not*4M | A/A* | 1.670 | (0.897, 2.960) | 16 | (6.4) | | |
| GRIK1 | rs2832407 | c1251 + 1338G > T | G/T | 0.998 | (0.712, 1.405) | 113 | (45.2) | 0.010577728 | 0.123857585 |
| | | | T/T | 0.561^{b} | (0.386, 0.813) | 67 | (26.8) | | |
| Intergenic | rs10494334 | 163535374G > A | G/A | 0.504^{b} | (0.335, 0.739) | 33 | (13.2) | 0.013114019 | 0.170482241 |
| | | | A/A | 0.519 | (0.122, 1.518) | 3 | (1.2) | | |
| CREBBP | rs3025684 | c3837 − 8C > T | C/T | 0.733 | (0.523, 1.016) | 56 | (22.4) | 0.015473778 | 0.216632886 |
| | | | T/T | 0.358^{b} | (0.172, 0.667) | 10 | (4.0) | | |
| CYP2D6 | rs3892097 | *4 1846G > A | C/T* | 1.690^{b} | (1.230, 2.308) | 74 | (29.6) | 0.017906734 | 0.304414473 |
| | | | T/T* | 1.940 | (0.966, 3.683) | 13 | (5.2) | | |
| DRD3 | rs6280 | c25G > A | G/A | 1.156 | (0.816, 1.645) | 92 | (36.8) | 0.017906734 | 0.303170461 |
| | | | A/A | 1.828^{b} | (1.283, 2.618) | 94 | (37.6) | | |
| GAL | rs694066 | c82 − 77G > A | G/A | 0.756 | (0.546, 1.036) | 61 | (24.4) | 0.017906734 | 0.293387275 |
| | | | A/A | 0.242^{b} | (0.073, 0.598) | 4 | (1.6) | | |
| CYP2D6 | rs28371706 | *17*40 1023C > T | G/A* | 0.511^{b} | (0.301, 0.824) | 19 | (7.6) | 0.018372619 | 0.330707144 |
| | | | A/A* | 0.256 | (0.041, 0.855) | 2 | (0.8) | | |
| DCC | rs12607853 | c*3949T > C | T/C | 0.712^{a} | (0.526, 0.963) | 91 | (36.4) | 0.025402346 | 0.504094938 |
| | | | C/C | 0.511^{b} | (0.329, 0.776) | 31 | (12.4) | | |
| GABRB3 | rs7165224 | c249 − 6417A > G | A/G | 0.431 | (0.178, 1.160) | 37 | (14.8) | 0.025402346 | 0.508046929 |
| | | | G/G | 0.793 | (0.347, 2.041) | 205 | (82.0) | | |
| TACR1 | rs6715729 | c333T > C | T/C | 1.013 | (0.698, 1.486) | 111 | (44.4) | 0.03403222 | 0.714676623 |
| | | | C/C | 0.627^{a} | (0.427, 0.927) | 88 | (35.2) | | |
| HTRB3 | rs1185027 | c*797T > A | T/A | 1.042 | (0.658, 1.691) | 115 | (46.0) | 0.034325907 | 0.755169948 |
| | | | A/A | 0.649 | (0.410, 1.053) | 106 | (42.4) | | |
| DRD2 | rs6275 | c939T > C | T/C | 1.642^{a} | (1.118, 2.456) | 120 | (48.0) | 0.036285498 | 0.884453066 |
| | | | C/C | 1.909^{b} | (1.275, 2.903) | 91 | (36.4) | | |
| OPRM1 | rs563649 | c291 − 2994C > T | C/T | 0.778 | (0.521, 1.136) | 36 | (14.4) | 0.036285498 | 0.907137455 |
| | | | T/T | 0.0 | (−635.96, 105.86) | 0 | (0.0) | | |
| TACR3 | rs1384401 | c888 + 12273C > T | C/T | 0.657^{a} | (0.473, 0.913) | 96 | (38.4) | 0.036285498 | 0.843321369 |
| | | | T/T | 0.569^{b} | (0.397, 0.812) | 67 | (26.8) | | |
| Intergenic | rs965972 | 193494720G > A | G/A | 1.505 | (0.687, 3.792) | 48 | (19.2) | 0.036776136 | 0.992955683 |
| | | | A/A | 2.314^{a} | (1.112, 5.638) | 194 | (77.6) | | |
| SORCS3 | rs728453 | c628 − 28349A > G | A/G | 0.641^{b} | (0.455, 0.889) | 53 | (21.2) | 0.036776136 | 0.959434255 |
| | | | G/G | 0.459 | (0.188, 0.958) | 7 | (2.8) | | |
| TACR3 | rs4530637 | 103585232A > G | A/G | 1.213 | (0.874, 1.686) | 92 | (36.8) | 0.048802999 | 1 |
| | | | G/G | 1.752^{b} | (1.228, 2.498) | 74 | (29.6) | | |

Human Genome Variation Society (HGSV); *Patient data presented as complimentary Strand.
^{a}= p < 0.05; ^{b}= <0.01; ^{c}Wild type information not included.

### Discussion

This study identified multiple genetic variants associated with OUD. As seen in FIG. 3, two dopamine reward pathway SNPs (rs324029 and rs2654754) within the single gene DRD3 were associated with an increased adjusted odds risk (OR) of OUD. Additionally, 2 SNPs (rs15524 and rs776746) within the drug metabolism gene CYP3A5 also displayed an increased adjusted OR for OUD. Conversely, CYP3A4 (rs2740574) and CYP1A2 (rs2069514), both genes involved in opioid metabolism, were associated with a decreased adjusted odds of OUD. The brain reward pathway has proven to be an incredibly strong driver of behavior, in which animals will ignore external rewards and accept painful stimuli in order to experience this reward. DRD3 regulates the expression of the dopamine 3 receptor and has demonstrated a role in drug seeking behavior and reward. The identified DRD3 rs324029 and rs2654754 found to be associated with an increased risk of OUD in this study are noteworthy considering prior findings of associations between DRD3 variants and heroin dependence. Although others have examined similar candidate SNPs, DRD3 rs324029 and rs2654754 were the only SNPs found to be consistent between this and previous studies. Differences in statistical analysis, allocation of patients into the control and OUD groups, and sample size may have contributed to the difference in findings. The identification of genetic variants associated with OUD provides the ability to advance care in multiple ways. First, these variants may inform the development of new drug targets for treatment of the disease. Methadone and buprenorphine are opioid agonists, whereas naltrexone is an opioid antagonist, all designed to decrease cravings for addictive substances. Second, PGx has been used broadly to inform the best treatment options for patients in medicine, ranging from the most effective cancer treatments to avoidance of medications with the potential to have adverse side effects. Genetic markers related to OUD can better inform the risk-benefit discussion when initiating or continuing opioid therapy.

Approximately one-third of medications that undergo oxidative metabolism are metabolized by CYP3A4/5. The rs15524 SNP found to be related to OUD in this study is not a component of any of the currently noted CYP3A5 star “*” alleles. Although not related to opioid metabolism, rs15524 (31611C>T (G>A)) is related to decreased drug metabolism (sirolimus) where individuals with the homozygous A/A (complementary T/T) genotype have associated higher drug concentrations, with resultant increased extent and duration of drug exposure compared with individuals with either the heterozygous G/A (complementary C/T) genotype or the homozygous G/G (complementary C/C) genotype. The rs776746 (6981A>G; complementary T>C) SNP of CYP3A5, also found to be associated with increased risk of OUD (OR 2.495 for C/C), is the defining variant seen in the CYP3A5*3 no function star allele. In 33 Japanese patients with cancer treated with transdermal fentanyl, CYP3A5*3/*3 group had significantly higher plasma fentanyl concentrations than the *1 allele group. Greater exposure to fentanyl was seen in patients homozygous for the CYP3A5*3 variant compared with the homozygous normal function form (CYP3A5*1/*1) or heterozygous normal/no function (CYP3A5*1/*3) form. CYP3A5 has been identified as a metabolism pathway for fentanyl, alfentanil, and methadone, however, the results of this study are the first to indicate a link between CYP3A5 and OUD, which is beyond the drug metabolism context.

Others have previously found that CYP3A4 (rs2242480) and CYP1A2 (rs3743484) were associated with a decreased risk of substance use disorder, and that at the gene level, CYP3A4 was associated with an increased risk of adverse reactions to codeine/tramadol and CYP1A2 was associated with poor pain control by codeine/tramadol in men. Reductions in CYP1A2 enzyme activity have also been associated with methadone toxicity. In the present study, however, there were no measurements of drug concentrations performed.

There are currently 35 identified star alleles of CYP3A4. Rs2740574 is one of many SNPs and is found in multiple suballeles of CYP3A4. Rs2740574 is the defining SNP of CYP3A4*1.001, a suballele of CYP3A4*1. Like other CYP3A4 star alleles, the function of CYP3A4*1 has not been assigned at this time. Rs2740574 has been related to the buprenorphine dose needed to prevent withdrawal symptoms, with heterozygous (T/C) or homozygous (C/C) individuals requiring higher doses to prevent withdrawal symptoms. CYP1A2 has 20 known variants. Rs2069514 is the definitive SNP of CYP1A2*1C. This SNP is related to decreased drug metabolism. As few prescription drugs are substrates for CYP1A2, little data exists relative to polymorphic CYP1A2 variants and drug metabolism. However, the A/A and A/G genotypes of rs2069514 are related to decreased metabolism of caffeine. Others have performed a genomewide association study (GWAS) to examine problematic opioid prescription use, opioid addiction, and OUD. It was found by others that two genomewide significant loci, KDM4A (rs3791033) and LRRIQ3 (rs640561), are associated with problematic opioid prescription use in 23andMe research participants of European ancestry. Others conducted a GWAS of the Genetics of Opioid Addiction Consortium (GENOA) data and found a correlation with OPRM1 (rs9478500) in those of European ancestry. Although eight variants of OPRM1 (rs1799971, rs1074287, rs510769, rs1381376, rs563649, rs9479757, rs2075572, and rs6848893) were included in the candidate SNPs in the study described in this example, OPRM1 (rs9478500) was not.

Others found OUD variant associations at OPRM1 (rs1799971 and rs79704991) and FURIN (rs11372849) in those with European ancestry and OPRM1 (rs9478500) in European and African ancestry groups. Due to the inaccuracies of self-reported race and being unable to determine genetic ancestries from the 180 preselected SNPs, it was not possible to complete an analysis accounting for the participants' genetic ancestry.

Inherently, in order to develop OUD, participants would have to have been exposed to opioids. However, some of the participants categorized as not meeting criteria for OUD also reported no prior opioid exposure. In the present study, the incidence rate of OUD identified among participants enrolled from the ED was high, at 19%. This incidence rate is attributed to the detailed screening utilized. SAMHSA reports the estimated national incidence rate of SUD is 0.6%. However, this estimation lacks those with unstable housing or in jail, which are populations known to have a higher incidence of OUD. ED visits for OUD have risen over the years and EDs in metropolitan areas care for a larger percentage of people with OUDs than those in other areas, where 64% of ED visits for OUD accounted for among 25% of all United States EDs. In addition, International Classification of Disease (ICD) codes have significant limitations when calculating actual incidence of this disease process, as it requires that the treatment team recognize and document it, as illustrated by the 64% of participants who were found to meet DSM-5 criteria for OUD in the Project ED Health (CTN0069) study and yet had no associated ICD 10 code recorded from the ED visit at which they were enrolled in the study. Natural language processing can enhance the number of patients identified as having OUD compared with ICD codes alone.

In conclusion, the identification of SNPs within the dopamine reward pathway (DRD3) and opioid metabolism (CYP3A5) pathway provides useful biomarkers for identifying patients at risk for developing OUD. Additionally, SNPS in CYP3A4 and CYP1A2 were associated with a decreased risk of developing OUD. The present findings indicate that these SNPs may serve as biomarkers for assessing the risk of developing OUD in patients, and may further be used to design customized treatments based on the assessed risk.

### Example II—Machine Learning Analysis

Self-reported factors potentially associated with OUD, including details of past opioid exposure, current indications of OUD, and other health history were collected. Patients responded to 277 survey questions. Results from this testing were integrated with the 180 candidate SNPs described in Example I above for application of an advanced machine learning algorithm ideal for processing such a large and complex data set. Results from the machine learning analysis (LASSO), shown in Table 6, identified 17 question areas and 1 SNP to be included in risk analysis modeling.

TABLE 6
LASSO variable identified as predictors of OUD

| Variable Name | Description | Response Options |
|---|---|---|
| Race | Self-reported | 1 - African American or Black |
| | | 2 - White |
| | | 3 - Asian |
| | | 4 - Native Hawaiian or Other Pacific Islander |
| | | 5 - American Indian or Alaska Native |
| | | 6 - Other Race |
| | | 7 - Biracial or Multiracial |
| hrs_per_week | How many hours per week do you | |
| | work? | |
| assist8_injection | Have you ever used any drug by | 0 - No, Never |
| | injection? | 1 - Yes, but not in the past 3 months |
| | | 2 - Yes, in the past 3 months |
| | | 3 - Not Answered |
| assist_tobacco_pos | Determination of positive abuse for | 0 - No |
| | tobacco on the ASSIST* | 1 - Yes |
| assist_amphetamines_pos | Determination of positive abuse for | 0 - No |
| | amphetamines on the ASSIST | 1 - Yes |
| assist_cocaine_pos | Determination of positive abuse for | 0 - No |
| | cocaine on the ASSIST | 1 - Yes |
| partner.substance.use | Composite measure of partner or | 0 - No |
| | cohabitant substance use | 1 - Yes |
| stress_severity | Severity of perceived stress | 0 - Low perceived stress |
| | | 1 - Moderate perceived stress |
| | | 2 - High perceived stress |
| | | 3 - Perceived stress not fully assessed |
| ptsd.screen | Composite measure from PTSD | 0 - symptoms not consistent with PTSD |
| | screening questions | 1 - symptoms consistent with PTSD |
| depression_severity | Depression severity | 0 - None to slight |
| | | 1 - Mild |
| | | 2 - Moderate |
| | | 3 - Severe |
| | | 4 - Anxiety was not fully assessed |
| anxiety_severity | Severity of anxiety based on | 0 - None to slight |
| | assessment score | 1 - Mild |
| | | 2 - Moderate |
| | | 3 - Severe |
| | | 4 - Anxiety was not fully assessed |
| pain_tx_gabapentin | Gabapentin/Neurontin, Lyrica/ | 0 - Never |
| | Pregabalin | 1 - Past 7 days |
| | | 2 - Ever |
| | | 3 - Prefer not to answer |
| od_life | Have you ever overdosed in your | 0 - No |
| | life? | 1 - Yes |
| | | 2 - Prefer not to answer |
| seen_od | In your lifetime, have you seen | 0 - No |
| | someone else overdose? | 1 - Yes |
| | | 2 - Prefer not to answer |
| treatment_hist | Have you ever gone anywhere or | 0 - No |
| | seen anyone for a reason that was | 1 - Yes |
| | related in any way to your use of | 2 - Prefer not to answer |
| | medicines or drugs - a physician, | |
| | counselor, Narcotics Anonymous, or | |
| | any other community agency or | |
| | professional? | |
| not_seek_help | Was there ever a time when you | 0 - No |
| | thought you should see a doctor, | 1 - Yes |
| | counselor, or other health | 2 - Prefer not to answer |
| | professional or seek any other help | |
| | for your drug use, but you didn't go? | |
| not_seek_help_other_opinion | Was there ever a time when someone | 0 - No |
| | else thought you should see a doctor, | 1 - Yes |
| | counselor, or other health | 2 - Prefer not to answer |
| | professional or seek any other help | |
| | for your drug use, but you didn't go? | |

| SNP Identified | Gene | Description |
|---|---|---|
| rs324029 | DRD3 | Regulation of the D3 dopamine receptor |

*ASSIST = Alcohol, Smoking, and Substance Involvement Screening Test from the Word Health Organization

Using these 17 questions and 1 SNP, a reliable risk assessment of an individual's likelihood of developing OUD can be made.

### Example III—Machine Learning Analysis of Candidate Genes and Behavioral Survey Data in Assessing OUD Risk

Machine learning was used to identify behavioral survey questionnaire items and SNPs associated with OUD.

Risk stratification of patients prior to an opioid prescription may improve overall pain management while reducing the risk of developing future OUD. The use of survey questionnaires or candidate gene SNPs individually to risk-stratify patients has been met with much scientific skepticism. To address this, machine learning technology was used to streamline and combine survey questions with six previously identified candidate gene SNPs. A total of 1,222 participants were included in the analyses for this study. Of included participants, 183 met the criteria for having OUD. The machine learning training was conducted from 122 participants with and 122 participants without OUD with remaining participants 917 for the validation set. Machine learning analysis reduced the survey questions from 123 to 9. The corresponding areas under the receiver operating characteristics curve (AUCs) were 0.88 and 0.89 for the use of the survey questions only or the survey questions plus the six SNPs, respectively. The sensitivity for the correct OUD classification was 62% for the survey questions only; however, the addition of the six SNPs improved this sensitivity to 77%. In sum, machine learning improves the prognostic reliability of using survey questions or candidate genes alone for determining the risk of OUD.

Example I describes six single SNPs from four genes that were associated with OUD and showed increased or decreased odds of OUD. These SNPs are rs15524 and rs776746 (related to CYP3A5) and rs324029 and rs2654754 (related to DRD3) (with which increased odds of OUD have been found), and rs2740574 (related to CYP3A4) and rs2069514 (related to CYP1A2) (with which decreased odds have been found). Although the level of evidence was moderate to low, a query of the Pharmacogenomics (PGx) Knowledgebase identified eight genes with 22 variants to be potentially linked to the increased risk of OUD and three to a decreased risk. Applying machine learning models to electronic health records has been used to predict the risk of OUD. However, the application of machine learning models to SNPs in candidate genes only for the prediction of OUD has been controversial.

In this example, a machine learning model was applied to a survey questionnaire derived primarily from the DSM-5 and WHO ASSIST 3.1 alone or in combination with the six candidate gene SNPs identified above as being associated with an increased or decreased risk of developing OUD.

### Results

### Machine Learning Analysis of the Behavioral Survey Questions in Assessing OUD Risk

In the training data, 52 variables had a significant association with OUD in univariate logistic regression after adjusting for false discovery rate. The list of variables was pruned to 36 after running regressions on the test data. Several variables were removed prior to stepwise selection due to nebulous causal association. The stepwise selection process selected 9 survey variables for model fit, and two variables (use of gabapentin and history of hospitalization for mental health disorders) had to be removed due to collinearity issues (Table 7).

TABLE 7
Survey variables

| | Response | |
|---|---|---|
| Variable Description | Options | Description of Association |
| Hours per week worked | | Each hour worked lowers the odds of OUD |
| Positive abuse for tobacco on the | 0 - No | Tobacco abuse on ASSIST increases the odds of |
| ASSIST | 1 - Yes | participant having OUD |
| Positive abuse for amphetamines on | 0 - No | Amphetamine abuse on ASSIST increases the odds |
| the ASSIST | 1 - Yes | of participant having OUD |
| Positive abuse for cocaine on the | 0 - No | Cocaine abuse on ASSIST increases the odds of |
| ASSIST | 1 - Yes | participant having OUD |
| Any sort of torso pain (from pain | 0 - No | Having any sort of torso pain increases the odds of |
| map) | 1 - Yes | having OUD |
| Any substance use among extended | 0 - No | Having an family member from the extended family |
| family (cousins, aunts, uncles, | 1 - Yes | with substance use issues increases the odds of |
| grandparents) | | having OUD |
| Use of pain patches or creams | 0 - No | Use of treatment pain patches increases the odds of |
| (lidocaine, NSAIDs, Voltaren/ | 1 - In the | having OUD relative to participants who did not; |
| Diclofenac) | past 7 days | those who used them more recently had a larger |
| | 2 - Ever | effect |
| | used | |
| | 3 - Prefer | |
| | not to | |
| | answer | |
| Witness someone overdose during | 0 - No | People who have seen overdoses have increased odds |
| lifetime | 1 - Yes | of having OUD |
| | 2 - Prefer | |
| | not to | |
| | answer | |
| Use of non-prescribed injection drugs | 0 - No | Use of non-prescribed injection drugs increases the |
| in lifetime | 1 - Yes | odds of having OUD |

### Classification Accuracy

The corresponding areas under the receiver operating characteristics curve (AUCs) were 0.85 and 0.86 for the use of the survey questions only or the survey questions plus the six SNPs, respectively. The sensitivity for the correct OUD classification was 62% for the survey questions only; however, the addition of the six SNPs improved this sensitivity to 76% (Table 8). The overall accuracy of the model with survey questions only was 76%, while the accuracy of the model with SNPs added was 80%.

TABLE 8
Outcomes

| | | | | non-OUD | OUD |
|---|---|---|---|---|---|
| | Sample | | | classification | classification |
| Outcome | Size | Model | AUC | (specificity) | (sensitivity) |
| OUD | n = 366 | Survey | 0.88 | 91% | 62% |
| | | questions only | | | |
| | | Survey + SNPs | 0.89 | 83% | 77% |

### Discussion

With this study, it was demonstrated that candidate gene SNPs and behavioral survey questionnaire profiling in conjunction with machine learning provides a prognostic tool for assessing OUD risk. The candidate gene study on 1,301 participants and identified 250 participants who met the DSM-5 criteria for OUD, described in Example I above, used logistic regression, adjusting for age and biologic sex, to characterize the association between each SNP and DSM-5 criteria consistent with OUD. From this analysis, the six SNPs utilized in the present study were identified. Homozygotic CYP3A5 (rs15524 and rs776746) and DRD3 (rs324029 and rs2654754) had odds ratios ranging from 1.5 to 2.8 for an increased risk of OUD, whereas homozygotic CYP3A4 (rs2740574) and CYP1A2 (rs2069514) had odds ratios ranging from 0.5 to 0.7 for a decreased risk of OUD. When these SNPs were combined with the machine learning behavioral survey questions, the sensitivity of the model improved from 62% to 77%, respectively.

Others have previously used 16 candidate gene SNPs to develop a predictive OUD risk model from patients diagnosed with and without OUD, and have then used the SNPs to develop a machine learning model. However, for those SNPs used, substantial allele frequency differences were found across ancestries and the five machine learning models used found that the 16 SNPs predicted ancestry better than OUD. Others have also applied machine learning methods to 15 of the original 16 SNPs to develop an OUD classifier (Yes or No) and demonstrated an 82.5% sensitivity and 79.9% specificity and found no differences with taking race and ethnicity into consideration. The six SNPs used in this example, however, were derived from the screening of 180 candidate SNPs, 120 of which were related to pharmacodynamics and the dopamine reward pathway and 60 of which were related to pharmacokinetics, specifically drug metabolism or transport in 1,301 emergency department patients. A secondary analysis of the influence of genetic variants related to OUD in individuals in the African American/Afro-Caribbean and European biogeographical ancestry groups (BGAG) and by sex was conducted. Because race/ethnicity are social constructs and ancestry is biological, the BGAG was used. From this analysis of the 6 SNPs used in this example, it was found that African American/Afro-Caribbean or European ancestry grouping, and sex, did not have an impact on OUD risk.

### Materials and Methods

### Study Design and Setting

This study involved cross-sectional data collection of randomly approached patients during an emergency department encounter (ED). Enrollment took place at two academic health systems encompassing three urban EDs in the Midwestern United States. The use of these data for this study was approved by the Institutional Review Board.

### Selection of Participants

A randomly generated selection of ED beds was chosen to determine order of persons to be approached in each ED treatment area, allowing for a representative sample. Patients were systematically approached for recruitment eligibility during their ED encounter between June 2020 and November 2021. All adult ED patients during recruitment hours who were not receiving active resuscitation efforts were considered for enrollment. Exclusion criteria were individuals less than 18 years of age, did not speak English, had guard present, were previously enrolled, physically restrained, or were not able to consent. Informed consent was obtained prior to data collection.

### Measurements

An in-depth research assistant guided survey questionnaire was used to collect demographics, assess for presence or current or past OUD, and screen for other factors that may contribute to OUD such as mental health conditions, other substance use and past life experiences. All participants were asked if they had used any opioids in the past 12 months, and if so whether they were prescribed and taken only as directed. Any participants who used opioids within the prior 12 months were further screened for symptoms of OUD with a survey checklist that was adapted from the DSM-5 criteria. Research assistants were trained by a clinical psychologist prior to study enrollment, and individuals with a score of 2 or higher were categorized as meeting criteria for symptoms of OUD. The second method of OUD symptom identification was based on the WHO ASSIST 3.1 criteria, including opioid use and other substances. The WHO ASSIST 3.1 has subscales to detect non-medical use of opioids (including prescription) as well as tobacco, nicotine (vaping), cannabis (including vaping), alcohol, cocaine, amphetamines, inhalants, sedatives, and hallucinogens. Participants were classified as moderate or higher risk for substance use problems if they scored 4 or greater or 11 or greater on the alcohol subsection.

### Genotyping

Cheek cell samples were shipped to Genemarkers LLC (Kalamazoo, MI) for genotyping of 180 SNPs theorized to be associated with OUD. The DNA was isolated and processed by Genemarkers according to known methods. However, only the six SNPs previously identified as being associated with OUD risk were used in the machine learning model.

### Statistical Analysis

Participants with complete data on survey questions and selected SNPs were used in this analysis; additionally, participants who had OUD in the past but not current OUD were dropped (n=1222). There were 183 (15.0%) participants who were classified as having OUD at the time of survey response.

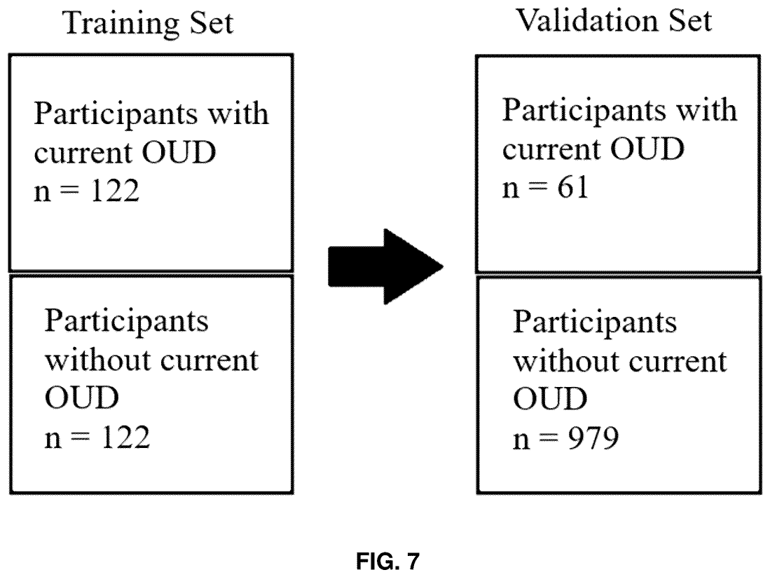

The dataset was split into training and validation sets to narrow the survey questionnaire. Two-thirds of participants with OUD were put in the dataset, and an equal number of participants without OUD were sampled at random without replacement. The remaining participants were used as a validation set (FIG. 7). Univariate logistic regression was performed for each survey question and p-values were adjusted using false discovery rate (FDR) adjustments. Only variables that had a significant association with OUD (FDR-adjusted p<0.05) passed into the validation pool. The process was repeated on the validation set to further refine the list of survey questions.

A balanced dataset was constructed by selecting all participants with OUD and randomly selecting an equal number of participants without OUD (n=366). This was done so model performance would not be biased toward the non-OUD participants. Some variables that passed filtering were dropped from the selection pool due to the vague causal direction between OUD and the survey question (e.g., did the participant have an overdose because they had OUD, or did the participant overdosing lead to the development of OUD?), a correlation being too high/basically a variable that confirms OUD (e.g., positivity on ASSIST scale for opioid use), or because including the variable in model building resulted in multicollinearity or singularity issues. Stepwise selection was used to create a multivariate logistic regression from the survey questions. Model performance was assessed with confusion matrices and ROC curves; sensitivity and specificity were derived from the confusion matrices while AUC was derived from the ROC curve.

### Example IV—Overdose Risk

A secondary analysis of the original study from the data in Example I above was conducted on the patients who experienced a non-fatal opioid overdose, to assess an association between SNPs and an elevated risk for having an opioid overdose. A multiple logistic regression analysis was conducted in the self-reported, non-fatal opioid overdoses of 1,301 adult patients seen in the emergency departments of three medical centers in Ohio. For this secondary analysis, a subsample of 821 patients reported having been exposed to opioids in their lifetime, 95 (11.6%) also reported having experienced an opioid-related overdose. Informed consent was obtained prior to data collection.

A detailed survey assessment to gather self-reported information on past opioid and other substance use, mental health disorders, and other health/medical history, and prior opioid overdose. Any participant who reported use of opioids within the past 12 months was then asked questions derived from the DSM-5 criteria for OUD to determine OUD.

### Genotyping

The cheek cell samples, via buccal swabbing, were shipped to a pharmacogenomics (PGx) testing laboratory (Genemarkers LLC, Kalamazoo, MI, USA) for genotyping related to 180 single nucleotide polymorphisms (SNP) thought to be associated with OUD. Of the 180 variants, 60 were related to pharmacokinetic pharmacogenes (metabolism and transport) and the remainder (120) were related to pharmacodynamic pharmacogenes with a potential association to the dopamine reward pathways.

### Statistical Analysis

Logistic regression, adjusting for age and biologic sex at birth, was used to characterize the association between each SNP and opioid overdose. For each categorical SNP, the reference group would be set as WT/WT. To control for multiple hypothesis testing, the Benjamini and Hochberg method called the False Discovery Rate (FDR) was utilized to determine statistical significance. For a more conservative understanding of which SNPs were associated while adjusting for age and sex, the Bonferroni Corrected p-value <0.05 was utilized for determining statistical significance. Ultimately, if the SNP had a Bonferroni Corrected p-value <0.05, it was deemed “statistically significant” and further investigated via their model diagnostics and visual representations. Finally, forest plots were generated to visually compare the effect sizes of statistically significant SNPs on a logarithmic scale, facilitating a comprehensive overview of the results.

### Demographic Data

Of the 1,301 participants in the original study, 95 participants reported having had experienced an opioid overdose. A majority of the 95 participants were of the European genetic ancestry group (80%) and were male (53.7%). Further, 71 (74.7%) of the 95 participants reporting having experienced an opioid overdose also met the criteria for OUD (Table 9).

TABLE 9
Patient characteristics of those having
experienced a non-fatal opioid overc

| | | Overdose | |
|---|---|---|---|
| N | | 95 | |
| Age (mean (SD)) | | 41.66 | (12.06) |
| Sex at birth (%) | Female | 44 | (46.3) |
| | Male | 51 | (53.7) |
| Race* (%) | European | 76 | (80) |
| | African American/Afro-Other | 15 | (15.8) |
| | | 4 | (4.2) |
| Non-OUD (%) | | 24 | (25.3) |
| OUD (%) | | 71 | (74.7) |

*Based on the standardized biogeographic grouping system.

Genetic Variants Associated with Opioid Overdose

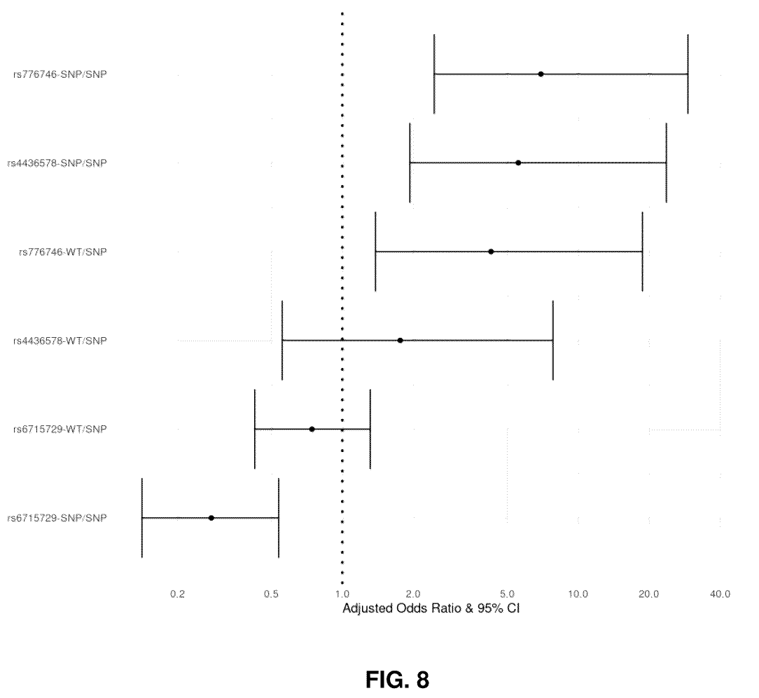

A total of four SNPs had FDR corrected p-values <0.05. Of those four SNPs, three SNPs Bonferroni corrected p-values <0.05. The estimates and 95% CI's which had Bonferroni corrected p-value <0.05 are found in Table 10. Three SNPs found in three genes were associated with opioid overdose: increased odds with CYP3A5 (rs776746) and DRD2 (rs4436578), and decreased odds with NKIR (rs6715729). Homozygotic CYP3A5 (rs776746) had the highest adjusted odds ratio (OR) of 6.96 (95% CI [2.45, 29.23]) and homozygotic NKIR (rs6715729) had the lowest OR of 0.28 (95% CI [0.14, 0.54) (FIG. 8). The one SNP that did not meet the Bonferroni correction cutoff but did meet the FDR cutoff of <0.05 was LINC01456 (rs2213602).

TABLE 10
SNPs associated with opioid overdose

| | | HGSV | Risk | Odds Ratio | | Bonferroni |
|---|---|---|---|---|---|---|
| | | Sequence | Geno- | (Confidence | | Corrected |
| Gene | rsID | Variant | type | Interval) | | p value |
| CYP3A5 | rs776746 | 6981A > G | T/C^{a} | 4.27 | (1.38, 18.75) | 0.00015 |
| | | | C/C | 6.96 | (2.45, 29.23) | 0.00015 |
| DRD2 | rs4436578 | c-31- | G/A | 1.76 | (0.56, 7.82) | 3.78E−06 |
| | | 11361G > A | | 5.58 | (1.93, 23.67) | 3.78E−06 |
| NK1R | rs6715729 | c333T > C | T/C | 0.74 | (0.43, 1.31) | 0.00015 |
| | | | C/C | 0.28 | (0.14, 0.54) | 0.00014 |

Human Genome Variation Society (HGSV); *Patient data presented as complimentary strand.
^{a}Patient data presented as complimentary strand.

Table 11 shows the false discovery rate for SNPs associated with OOD.

| | | | Risk | Odds Ratio | |
|---|---|---|---|---|---|
| | | HGSV | Geno- | (Confidence | |
| Gene | rsID | Sequence Variant | type | Interval) | FDR |
| LINC01456 | rs2213602 | g.18089955A > G | A/G | 0.61 (0.25, 1.36) | 0.0012 |
| | | | G/G | 0.33 (0.16, 0.61) | 0.0012 |

### Discussion

Three genetic variants associated with opioid overdose were identified. CYP3A5 (rs776746) and DRD2 (rs4436578) were associated with an increased adjusted odds ratio of opioid overdose. Conversely, NK1R (rs6715729) was associated with a decreased adjusted odds of opioid overdose.

The finding that CYP3A5 (rs776746) was associated with an increased risk of opioid overdose is consistent with IMFs contributing most significantly to opioid overdoses. Notably, the SNP rs776746 is also one of the SNPs which is predictive of an increased risk of OUD, as shown in Example I above. It is indeed surprising that OUD and opioid overdose likelihood may be associated with the same gene. CYP3A5 has been identified as a metabolism pathway for the opioids fentanyl, alfentanil, and methadone. Rs776746 is the definitive SNP defining the no function/loss of function CYP3A5*3 allele. Loss of function SNPs in CYP3A5 has been associated with increased plasma concentrations of fentanyl. Significantly higher plasma fentanyl concentrations were associated with CYP3A5*3/*3 than the *1 allele group in 33 Japanese cancer patients treated with transdermal fentanyl. In a similar fashion, fentanyl plasma concentrations were higher in patients homozygous for the CYP3A5*3 variant compared to the homozygous normal function form (CYP3A5*1/*1) or heterozygous normal/no function (CYP3A5*1/*3) form.

DRD2 regulates the expression of the dopamine receptor 2 (D2R). A review of the PharmGKB database revealed the DRD2 (rs12364283) is associated with an increase risk of heroin dependence. Dopamine release in the nucleus accumbens is associated with the rewarding effects of opioids. DRD2 (rs4436578) has also been associated increased risk of heroin dependence. Here, an association between DRD2 (rs4436578) and risk for opioid overdose was found. Because of the role of dopamine in schizophrenia, DRD2 (rs4436578) has been primarily studied in this population. The present findings indicate that DRD2 (rs4436578) is a gene useful for assessing opioid overdose risk.

The neurokinin 1 receptor (NK1R) is a GPCR co-localized with the mu opioid receptor in the preBotzinger Complex within the medulla. The NK1Rs are important for maintaining rhythmic breathing which is essential for sustaining life. NK1R antagonists have been shown to reduce opioid adverse reactions. In the present study, it was found that NK1R (rs6715729) was associated with a decreased risk of opioid overdose. To date, NK1R (rs6715729) has not been well studied for its role in opioid-induced respiratory depression (OIRD). However, given the important role of the NK1R in OIRD, the present finding of a decreased risk of opioid overdose with NK1R (rs6715729) is consistent with the believed physiological role of NK1R in controlling respiration.

In conclusion, the identification of SNPs within the dopamine reward pathway (DRD2) and opioid metabolism (CYP3A5) pathway provides useful biomarkers for identifying patients at risk of opioid overdose. Further, the SNP within the NK1R (a regulator of OIRD) has the ability to be used as a marker for a decreased risk of OIRD and as a therapeutic target in preventing opioid overdose.

Certain embodiments of the kits and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the kits and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method of diagnosing a likelihood of opioid use disorder (OUD) in a patient, the method comprising:
(a) obtaining a biological sample from the patient;
(b) isolating DNA from the biological sample;
(c) genotyping the DNA for one or more single nucleotide polymorphisms (SNPs), wherein the one or more SNPs include rs15524, rs776746, rs324029, rs2654754, rs2740574, or rs2069514;
(d) determining the presence or absence of the one or more SNPs in the isolated DNA; and
(e) diagnosing the patient's likelihood of developing OUD based on the presence or absence of the one or more SNPs, wherein:
a presence of any of rs15524, rs776746, rs324029, or rs2654754 is indicative of an increased risk of developing OUD, and
a presence of either of rs2740574 or rs2069514 is indicative of a decreased risk of developing OUD.

2. The method of claim 1, further comprising:
(f) administering to the patient a non-opioid pain management treatment if the patient has any of rs15524, rs776746, rs324029, or rs2654754, or administering to the patient an opioid for pain management if the patient has either of rs2740574 or rs2069514.

3. The method of claim 2, wherein the non-opioid pain management treatment is a non-opioid analgesic.

4. The method of claim 2, wherein the non-opioid pain management treatment is physical therapy, cognitive behavioral therapy, or a combination thereof.

5. The method of claim 1, further comprising:
(f) administering to the patient an opioid agonist or opioid antagonist if the patient has any of rs15524, rs776746, rs324029, or rs2654754.

6. The method of claim 5, wherein the opioid agonist is methadone or buprenorphine.

7. The method of claim 5, wherein the opioid antagonist is naltrexone, nalmefene, or naloxone.

8. The method of claim 5, further comprising administering an opioid to the patient.

9. The method of claim 1, wherein the one or more SNPs are a combination of at least two of rs15524, rs776746, rs324029, rs2654754, rs2740574, and rs2069514.

10. The method of claim 1, wherein the absence of each of rs15524, rs776746, rs324029, and rs2654754 is indicative of no elevated risk of developing OUD.

11. The method of claim 1, wherein the absence of both of rs2740574 and rs2069514 is indicative of no decreased risk of developing OUD.

12. The method of claim 1, further comprising administering a treatment to the patient based on the patient's diagnosed likelihood of developing OUD.

13. The method of claim 1, further comprising asking the patient diagnostic questions.

14. The method of claim 5, further comprising adjusting a dosage of the opioid agonist based on the patient's genotyping results and odds ratio for OUD for rs15524, rs776746, rs324029, rs2654754, rs2740574, and rs2069514, wherein the dosage is decreased when any of rs15524, rs776746, rs324029, or rs2654754 is present, and the dosage is increased when either of rs2740574 or rs2069514 is present.

15. A diagnostic kit for diagnosing a likelihood of a subject developing opioid use disorder (OUD) based on the presence or absence of one or more single nucleotide polymorphisms (SNPs), the diagnostic kit comprising:
(a) a set of primers or probes for genotyping two or more of rs15524, rs776746, rs324029, rs2654754, rs2740574, and rs2069514 in DNA extracted from a biological sample; and
(b) instructions for using the primers or probes to diagnose the subject's likelihood of developing OUD based on the presence or absence of the SNPs.

16. A method of determining a subject's likelihood of developing opioid use disorder (OUD), the method comprising:
obtaining a biological sample from the subject;
isolating DNA from the biological sample;
genotyping the DNA for the presence of a homozygous single nucleotide polymorphism (SNP) in the CYP3A5 gene or the DRD3 gene;
determining whether the homozygous SNP in the CYP3A5 gene or the DRD3 gene is present in the DNA; and
determining the subject is likely to develop OUD if the homozygous SNP in the CYP3A5 gene or the DRD3 gene is present in the DNA.

17. The method of claim 16, wherein the homozygous SNP in the CYP3A5 gene is either rs15524 or rs776746.

18. The method of claim 16, wherein the homozygous SNP in the DRD3 gene is either rs324029 or rs2654754.

19. The method of claim 16, further comprising administering to the subject a non-opioid pain management treatment.

20. A method of predicting a likelihood of a subject experiencing an opioid overdose, the method comprising:
obtaining a biological sample from the subject;
isolating DNA from the biological sample;
genotyping the DNA for single nucleotide polymorphisms (SNPs) comprising rs776746, rs4436578, and rs6715729;
determining the presence or absence of the SNPs in the DNA; and
diagnosing an increased risk of the subject experiencing an opioid overdose if rs776746 or rs4436578 is present in the DNA, and diagnosing a decreased risk of the subject experiencing an opioid overdose if rs6715729 is present in the DNA.

21. The method of claim 20, further comprising treating the subject with a non-opioid treatment or a reduced dose of an opioid.
